(19) United States
(12) Patent Application Publication
(10) Pub. No.: US 2021/0022664 A1
(43) Pub. Date: Jan. 28, 2021

(54) NON-INVASIVE STAGING OF PRESSURE ULCERS USING PHOTOACOUSTIC IMAGING

(71) Applicant: The Regents of the University of California, Oakland, CA (US)

(72) Inventors: Jesse Jokerst, La Jolla, CA (US); Ali Hariri, La Jolla, CA (US)

(21) Appl. No.: 16/937,829

(22) Filed: Jul. 24, 2020

Related U.S. Application Data
(60) Provisional application No. 62/877,999, filed on Jul. 24, 2019.

(51) Int. Cl. A61B 5/00; A61G 10/02

(52) U.S. Cl. CPC A61B 5/447; A61G 10/026; A61B 5/0095

(57) ABSTRACT

A method of identifying a subdermal feature in a subject includes directing optical energy into a subject to photoacoustically generate ultrasonic waves from a dermal or subdermal feature in tissue. Signals representing ultrasonic waves that are generated from the dermal or subdermal feature are received. The signals representing the ultrasonic waves are processed to generate image data of the dermal or subdermal feature. It is determined from the image data that the dermal or subdermal feature is a non-healing skin lesion selected from the group including a pressure ulcer, a diabetic foot ulcer, an arterial insufficiency injury, a decubitus ulcer, a diabetic ulcer, and an insufficiency injury.

### BACKGROUND TO RELATED APPLICATIONS

This application claims the benefit of U.S. Provisional Application No. 62/877,999, filed Jul. 24, 2019, the contents of which are incorporated herein by reference.

### BACKGROUND

Pressure ulcers and diabetic foot ulcers are a pervasive and expensive health care challenge. The Healthcare Research and Quality Agency recently estimated that pressure ulcers exert an annual burden of $9.1-$11.6 billion on the United States' healthcare system. Pressure ulcers are defined by both the depth of ulceration and types of tissue affected. In stage I, the epidermis appears reddened and is characterized by non-blanchable erythema under light pressure. stage II is defined by ruptured skin and a loss of the epidermis and dermis, i.e. the formation of a visual ulcer.

Stage III involves full skin loss—the lesion extends to subcutaneous tissue. Whole skin loss, muscle necrosis, and damage to tendons and joints occur in stage IV. Ulcernecrosis is attributed to the loss of blood flow under sustained pressure. Visual inspection is the standard-of-care for triaging pressure ulcers. In some cases, pressure-sensitive devices are used to alert medical staff when a certain pressure threshold is exceeded. However, this threshold value is patient-dependent and varies with physiological metrics such as body mass index. The hematron sensor can measure the thermal conductivity of skin, which is correlated to blood flow. However, this sensor cannot monitor the progressive tissue damage that characterizes ulcer development.

One of the most well-known methods for pathology detection and tissue characterization is ultrasound elastography. This method extends the typical information supplied by ultrasound—a real time, affordable, and non-invasive modality. Ultrasound elastography is considered an intrinsic factor evaluation to estimate the stiffness of tissue by measuring strain making it applicable to detection of pressure ulcers. This method is limited by superficial tissue assessment, artifacts from compression, and examiner dependency. Nixon et al. utilized laser Doppler imaging to validate the clinical grading of erythema in 37 pressure ulcer patients and showed that this method could monitor a range of blood flow values for normal skin and areas of erythema. However, the laser Doppler imaging method suffers from low penetration depth and therefore monitoring the injury on underlying tissues is impossible. Recently, Swisher et al. used flexible electrode arrays to measure the impedance correlated with tissue health and wound types in a rat model. However, this device is not able to measure the effect of ulcers on subcutaneous fat tissue and muscle. None of the above methods can monitor the sub-dermal extent of pressure damage in real-time. If damage to dermal or sub-dermal tissue could be identified before it is visible by eye, then ulcer-associated morbidity and costs could be prevented before they incur. Therefore, improved methods for detecting the extent of ulceration beneath the skin are of significant interest.

### SUMMARY

Ulcers, including pressure ulcers and diabetic foot ulcers, damage the skin and underlying tissue in people with compromised blood circulation. They are classified into four stages of severity and span from mild reddening of the skin to tissue damage and muscle/bone infections. In accordance with one aspect of the subject matter described herein, we used photoacoustic imaging as a non-invasive method for detecting early tissue damage that cannot be visually observed while also staging the disease using quantitative image analysis. We used a mouse model of pressure ulcers by implanting sub-dermal magnets in the dorsal flank and periodically applying an external magnet to the healed implant site. The magnet-induced pressure was applied in cycles, and the extent of ulceration was dictated by the number of cycles. We used both laser- and LED-based photoacoustic imaging tools with 690 nm excitation to evaluate the change in photoacoustic signal and depth of injury. Using laser-based photoacoustic imaging system, we found a 4.4-fold increase in the photoacoustic intensity in stage I versus baseline (no pressure). We also evaluated the depth of injury using photoacoustics. We measured a photoacoustic ulcer depth of 0.38±0.09 mm, 0.74±0.11 mm, 1.63±0.4 mm, and 2.7±0.31 mm (n=4) for stages I, II, III, and IV, respectively. The photoacoustic depth differences between each stage were significant (p<0.05). We also used an LED-based photoacoustic imaging system to detect early stage (stage I) pressure ulcers and observed a 2.5-fold increase in photoacoustic signal. We confirmed the capacity of this technique to detect dysregulated skin even before stage I ulcers have erupted. We also observed significant changes in photoacoustic intensity during healing, suggesting that this approach can monitor therapy. These findings were confirmed with histology.

In one particular aspect of the subject matter described herein, a method of identifying a subdermal feature in a subject is provided. The method includes directing optical energy into a subject to photoacoustically generate ultrasonic waves from a dermal or subdermal feature in tissue. Signals representing ultrasonic waves that are generated from the dermal or subdermal feature are received. The signals representing the ultrasonic waves are processed to generate image data of the dermal or subdermal feature. It is determined from the image data that the dermal or subdermal feature is a non-healing skin lesion selected from the group including a pressure ulcer, a diabetic foot ulcer, an arterial insufficiency injury, a decubitus ulcer, a diabetic ulcer, and an insufficiency injury.

In another particular aspect of the subject matter described herein, the determining includes determining from the image data that the dermal or subdermal feature is a non-healing skin lesion before the non-healing skin lesion is visible by eye.

In another particular aspect of the subject matter described herein, the determining includes determining that the non-healing skin lesion selected from the group including a pressure ulcer, a diabetic foot ulcer, an arterial insufficiency injury, a decubitus ulcer, a diabetic ulcer, and an insufficiency injury extends beyond a region that is visible by eye.

In another particular aspect of the subject matter described herein, the method further includes staging the non-healing skin lesion based at least in part on an effective depth of the subdermal feature.

In another particular aspect of the subject matter described herein, when determining that the dermal or subdermal feature is a pressure ulcer, the method further includes staging the pressure ulcer based at least in part on an effective depth of the subdermal feature.

In another particular aspect of the subject matter described herein, staging the pressure ulcer is based at least in part on a photoacoustic intensity of the image data.

In another particular aspect of the subject matter described herein, a later stage is assigned to the pressure ulcer above a baseline as the effective depth of the subdermal feature increases.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1 illustrates the experimental procedure using a rodent model of human pressure ulcers.

FIGS. 2A(i), 2A(ii), 2A(iii), and 2A(iv) are photographs of the ulcer sites in mice at stages I, II, III, and IV, respectively; FIG. 2B represents the average histogram of five B-mode baseline fields-of-view for four animals; FIGS. 2C, 2E, 2G, and 2I show photoacoustic/ultrasound images with progression through stages I, II, III, and IV respectively; and FIGS. 2D, 2F, 2H, and 2J are representative photoacoustic A-line profiles (dotted line), which correspond to FIGS. 2C, 2E, 2G, and 2I, respectively.

FIG. 3A illustrates the photoacoustic intensity as a function of ulcer stage; FIG. 3B quantifies the photoacoustic depth effect of the pressure ulcer on the skin and underlying tissues at different stages; FIG. 3C shows ultrasound/photoacoustic images at 0, 30, 60, and 90 minutes after stage I to simulate healing/therapy; and FIG. 3D shows the photoacoustic intensity as a function of healing time and thus quantifies the photoacoustic intensity effect of healing procedure.

FIG. 4A shows a B-mode ultrasound image at baseline conditions when no pressure has been applied; FIG. 4B shows a B-mode photoacoustic image at baseline at the same position as in FIG. 4A; FIG. 4C shows a B-mode photoacoustic/ultrasound overlay at baseline conditions; FIG. 4D shows a B-mode ultrasound image at stage I; FIG. 4E shows a B-mode photoacoustic image at stage I at the same position as in FIG. 4D; and FIG. 4F shows a B-mode photoacoustic/ultrasound overlay at stage I.

FIG. 5A shows the experimental procedure used to monitor pre-stage I ulcers; and FIG. 5B shows the photoacoustic intensity at each cycle between baseline and stage I.

FIGS. 6A and 6B are histological images of the skin and underlying tissue, respectively, for the baseline condition (no pressure ulcer); FIG. 6C shows a histology image of the skin at stage I; FIG. 6D shows the histology of muscle for the animals with stage I ulcers; FIGS. 6E and 6F show histological images of skin and muscle, respectively, at stage II; FIG. 6G shows a histological image of skin in stage III of the pressure ulcer; FIG. 6H shows a histological image of muscle at stage III of the pressure ulcer; FIG. 6I shows the histological image of skin in stage IV; and FIG. 6J shows a histological image of muscle at stage IV showing large necrotic regions of muscle.

FIG. 7A shows a photograph (i) and photoacoustic 3D mapping (ii) of an arterial insufficiency injury located at the left ankle (tibio-talar) joint in a patient; FIG. 7B presents a quantitative analysis of the arterial insufficiency injury and healthy adjacent tissue showing a statistically significant increase in photoacoustic signal intensity at 690 nm, 850 nm, 690 nm and 850 nm LED wavelengths; and FIG. 7C shows a column average plot that was performed on all framed images and averaged to analyze mean photoacoustic signal intensity in the wound bed.

### DESCRIPTION

Photoacoustic imaging is a non-invasive and high-resolution technique that combines optical and ultrasound imaging features. This hybrid imaging modality offers higher penetration depth with less scatter than optical imaging. Nanosecond or picosecond optical pulses fired into tissue launch thermo-elastic-induced acoustic waves which are detected and reconstructed to form high-resolution images. As discussed in more detail below, we have found that the dysregulated vasculature associated with ulcers could be imaged using photoacoustic imaging to study their development and predict eruption. In particular, photoacoustic imaging is used to detect early-stage pressure ulcers and monitor their development across different stages using an established murine model.

Animal Model and Validations

The techniques described herein were first validated using an animal model. Twenty-five nude mice (8-10 weeks, 25-35 g) were kept in separate cages under a 12-hour light-dark cycle and sterile environment at constant temperature and humidity. All animal experiments were performed in accordance with NIH guidelines and approved by the Institutional Animal Care and Use Committee (IACUC) at the University of California, San Diego.

For the surgical implantation of the internal magnet (diameter of 7 mm and thickness of 1 mm, K&J Magnetic, Inc.), mice were anesthetized with 1-2% isoflurane and placed on a heating bed. The magnet was sterilized via autoclave. The skin was disinfected with povidone iodine at the surgical site. After creating a 5-mm incision, we placed the sterile magnet under the greater gluteus muscle and away from the incision. The incision was sealed using topical tissue adhesive (Abbott Laboratories, IL, USA). We waited ten days for healing to ensure that the surgical wound did not interfere with the pressure induction site. A sterile external magnet (identical to the internal magnet) was then placed on the skin surface above the implant site at regular intervals. The external magnet was applied in cycles. Each cycle consisted of 2 hours of magnet-induced pressure followed by 1 hour of release.

Wassermann E, Van Griensven M, Gstaltner K, Oehlinger W, Schrei K, Redl H, “A chronic pressure ulcer model in the nude mouse,” Wound Repair and Regeneration 2009; 17(4):480-4, showed in their model that 4, 6, 8, and 10 cycles were needed to create stage I, II, III, and IV ulcers, respectively, and we followed this protocol. Imaging was performed either between cycles or between stages. Three mice were sacrificed at baseline conditions and after each stage for histology. The skin and underlying tissue were fixed in buffered 10% formaldehyde solution. Histological analysis (H&E staining) was performed to stage the ulcer as an independent method.

Photoacoustic Imaging

We used both laser and LED-based photoacoustic imaging systems to perform all in vivo procedures. The Vevo LAZR (VisualSonic Inc.) is a laser integrated high frequency ultrasound system that utilizes a linear array transducer (LZ-550, Fc=40 MHz) with optical fibers integrated to both sides of the transducer. For optical excitation, this system uses a Q-switched Nd:YAG laser (4-6 ns pulse width) with a repetition rate of 20 Hz (frame rate of 6 Hz) followed by an optical parametric oscillator (tunable wavelength 680-970 nm). The laser intensity on the surface of the skin was measured at 12.65±0.65 mJ/cm2 using a laser pyroelectric energy sensor (PE50BF-C, Ophir LLC, USA). We also used a LED-based photoacoustic imaging system (CYBERDYNE Inc.). The system is equipped with a 128-element linear array ultrasound transducer with a central frequency of 10 MHz and a bandwidth of 80.9% fitted with two 690 nm LED arrays. (The repetition rate of these LEDs is tunable between 1, 2, 3, and 4 K Hz. The pulse width can be changed from 50 ns to 150 ns with a 5-ns step size. The LED intensity at 4 K Hz on the surface of skin was measured at 5.5 μl/cm2 using a photodiode sensor (S120C, Thorlabs Inc., USA). In both systems, the transducer can be scanned to generate three-dimensional (3D) images using a maximum intensity projection (MIP) algorithm.

LED-based photoacoustic imaging systems have a number of important advantages over laser-based systems, particularly when it is to be used in a clinical setting. Laser-based photoacoustic imaging systems are generally bulky, expensive and suffer from wavelength and power intensity fluctuations. They also typically use a laser enclosure system to shield the operator from irradiation, but this is cumbersome and prevents the operator from directly interacting with the subject

LED-based photoacoustic imaging systems, on the other hand, solve many of these issues in the clinical translation of photoacoustic imaging. For instance, they offer a significant reduction in cost, are significantly more stable than many laser-based systems and occupy only a fraction of the space. This stability and reduced footprint allow LED-based photoacoustic imaging systems to be portable. Furthermore, LEDs do not have to be in a light-tight enclosure, which can restrict many clinical procedures.

Quantitative and Statistical Analysis

We collected B-mode photoacoustic/ultrasound images from five different positions above the target site in each animal before initiating the pressure ulcer model (baseline conditions) and after each stage. Each B-mode image contains 256 A-line scans (an A-line scan is simply a single line of collected data). We used 690 nm as the excitation wavelength for all scans. We analyzed our data using two different categories: photoacoustic intensity and photoacoustic depth. The intensity was quantified by first converting all photoacoustic data to 8-bit images; the mean values and standard deviations of the accumulative photoacoustic pixel intensities from 3 mm×3 mm regions of interest (ROI; each ROI contained 50 A-line scans) were measured using ImageJ (Bethesda, Md., USA).

To quantitate the extent of disease, photoacoustic depth was calculated via 10 A-line intensity profiles from each ROI (every fifth A-line) on all photoacoustic images in all four animals. For the photoacoustic images, we measured the average and standard deviation of ulcer depths from ten different A-lines. The error bars in each figure represent the standard deviation from 4 different animals, and P values lower than 0.05 were considered significantly different.

Results

Pressure ulcers and diabetic foot ulcers are known to dysregulate the vasculature of tissue, but this dysregulation is often difficult to detect until the lesion has advanced to stage II or III where it has erupted through the skin. Thus, we hypothesized that photoacoustic imaging could be used to detect early stage lesions because it quantitates tissue absorption including from hemoglobin and deoxyhemoglobin. This early-stage detection of pressure ulcers could then be used to direct treatment and prevent the ulcer from progressing further and disrupting the epithelial barrier—these ruptured lesions are what cause the long-term complications.

We used a rodent model of human pressure ulcers to validate this imaging modality. There are several available animal models of pressure ulcers, including swine and rats. They usually apply pressure on the skin against the underlying bone; however, the use of anesthesia during this long procedure is the main limitation of this method. This model is limited to stage I ulcers due to the superficial position of the steel plate under the skin. Wassermann et al. introduced a chronic pressure model to induce all four stages by implanting a magnet under both the skin and deeper tissue layers, including muscle. Their model was verified with histology.

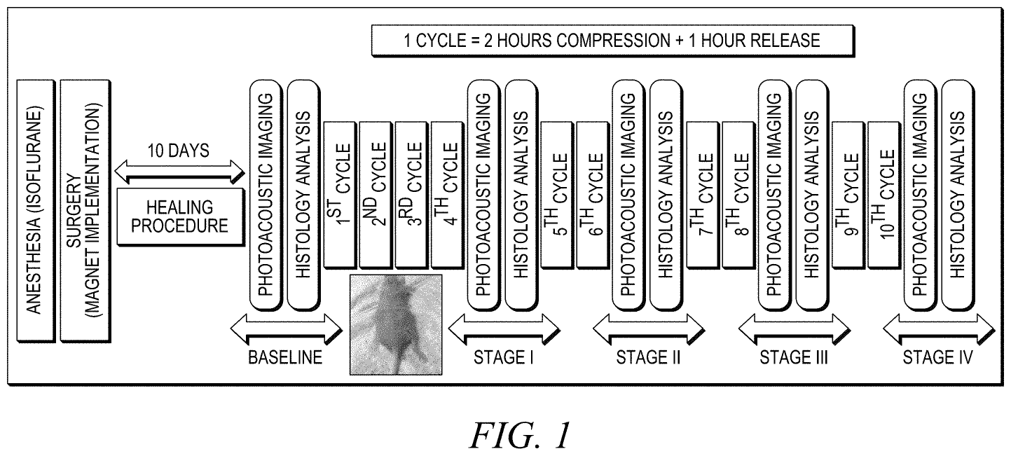

The experimental procedure using this model is illustrated in FIG. 1. All animals were anesthetized with 1-2% isoflurane and placed on a heating bed. The animals were allowed to heal for 10 days after implanting the internal magnet to ensure that the incision did not interfere with the pressure induction site. To create stage I, II, III, and IV pressure ulcers, 4, 6, 8, and 10 cycles were applied, respectively. Each cycle included 2 hours of pressure followed by 1 hour of release. Photoacoustic/ultrasound images were acquired at baseline and at each stage with a 40 MHz transducer. Histology analysis (H&E staining) was used to confirm ulcer stage.

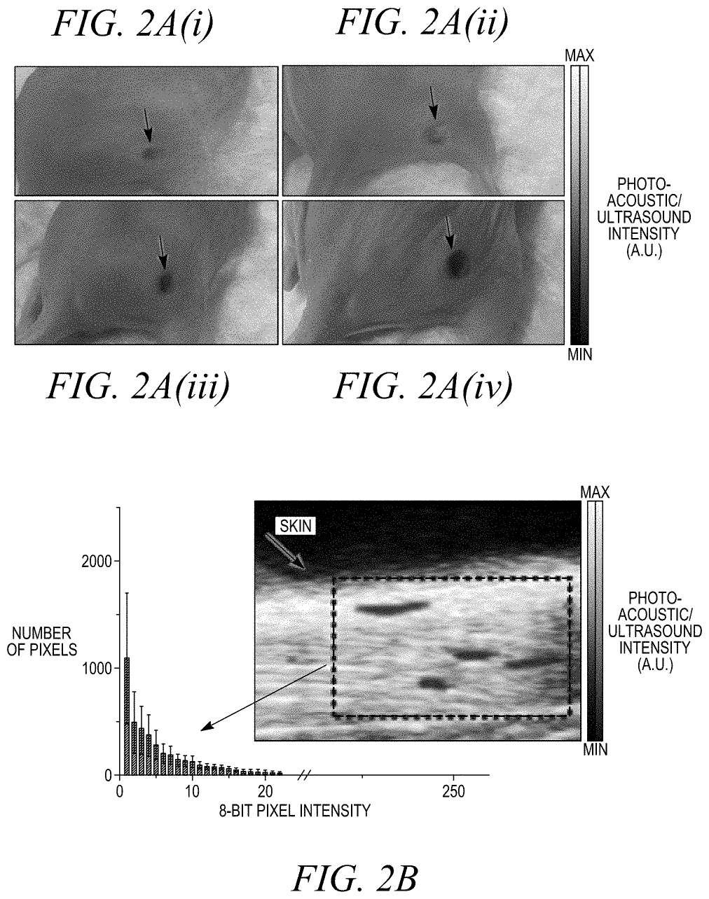

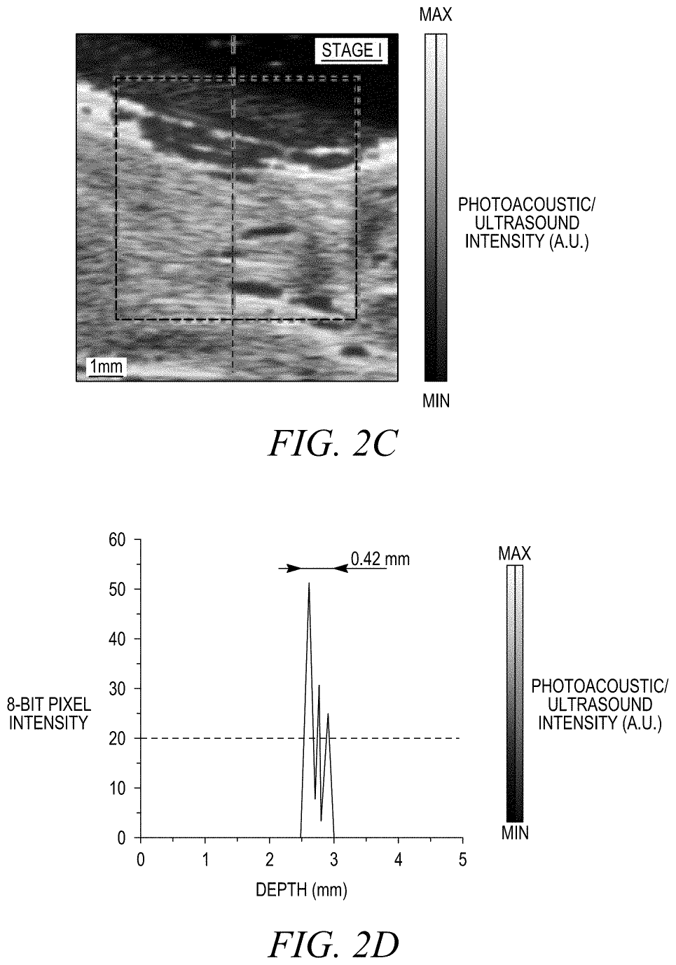

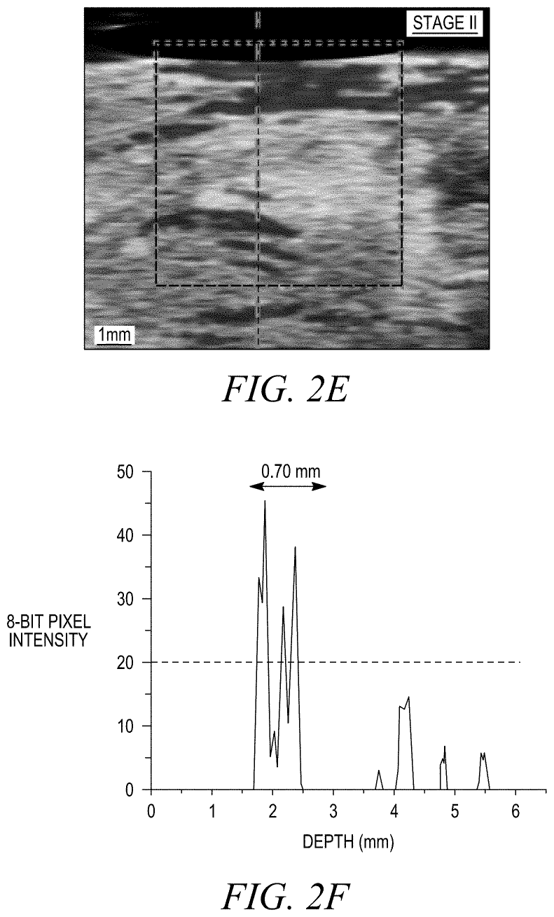

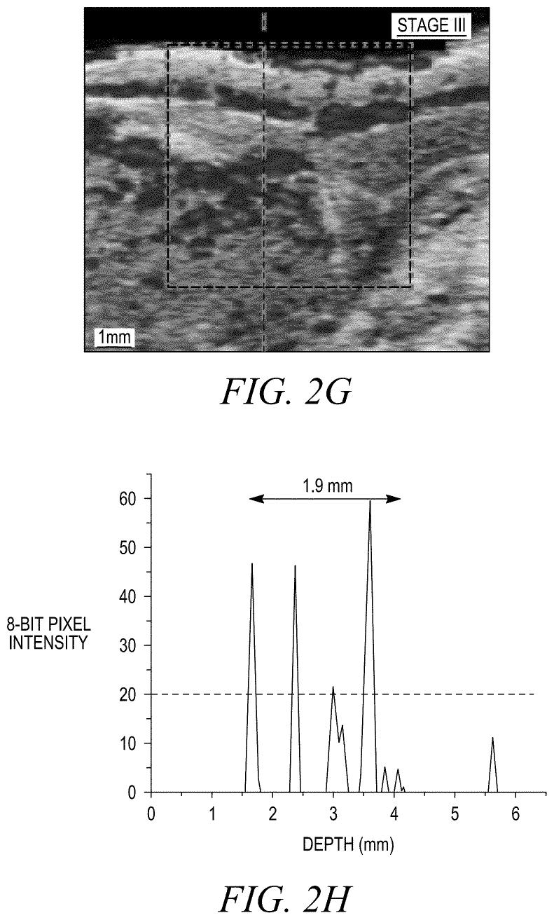

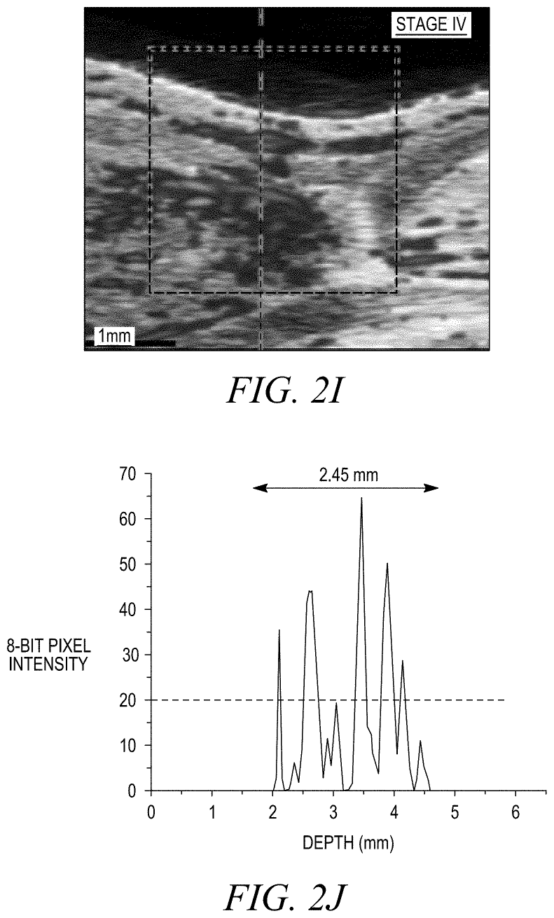

Our first goal was to confirm that the model was created correctly. FIGS. 2A(i), 2A(ii), 2A(iii), and 2A(iv) are photographs of the ulcer sites in mice at stages I, II, III, and IV, respectively. These photographs show the effect of ulcers on the skin but not the underlying tissues. These same animals were then studied with ultrasound/photoacoustic imaging to evaluate the wound depth. FIG. 2B represents the average histogram of five B-mode baseline fields-of-view for four animals. All average pixel intensities are lower than 20, and thus we used this number as the threshold for detecting the presence of ulcers using photoacoustic imaging.

The inset in FIG. 2B is a baseline photoacoustic/ultrasound image. FIGS. 2C, E, G, and I show the photoacoustic/ultrasound image with progression through stages I, II, III, and IV respectively. FIGS. 2D, F, H, and J are representative photoacoustic A-line profiles (dotted line), which correspond to FIGS. 2C, E, G, and I, respectively. Note that the full analysis used 10 line profiles per image. We considered pixels with an 8-bit photoacoustic intensity >20 to represent ulceration of the underlying tissues.

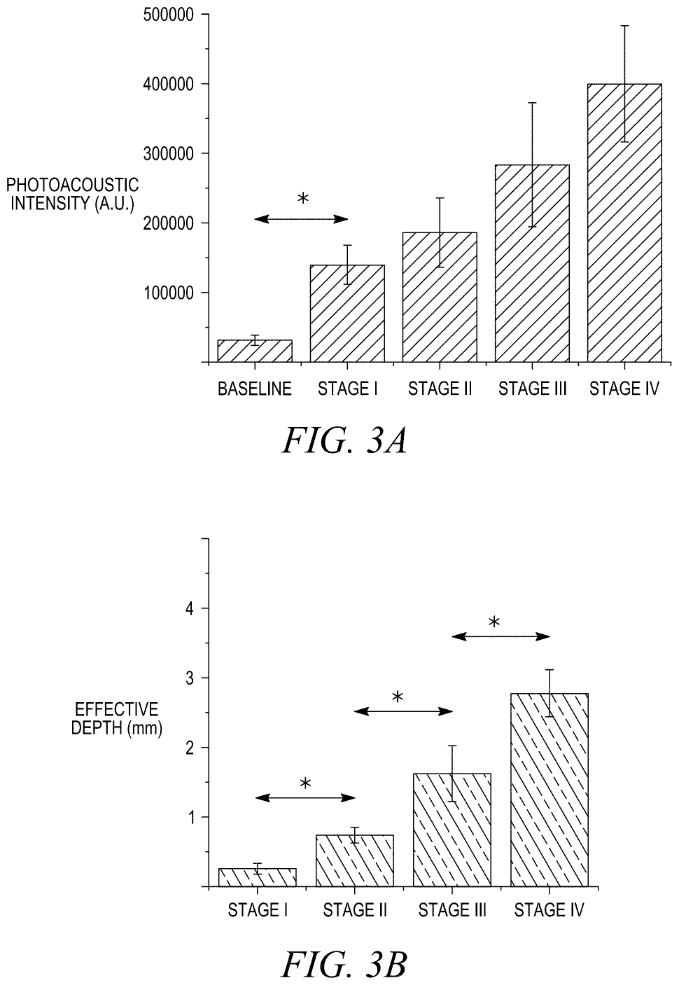

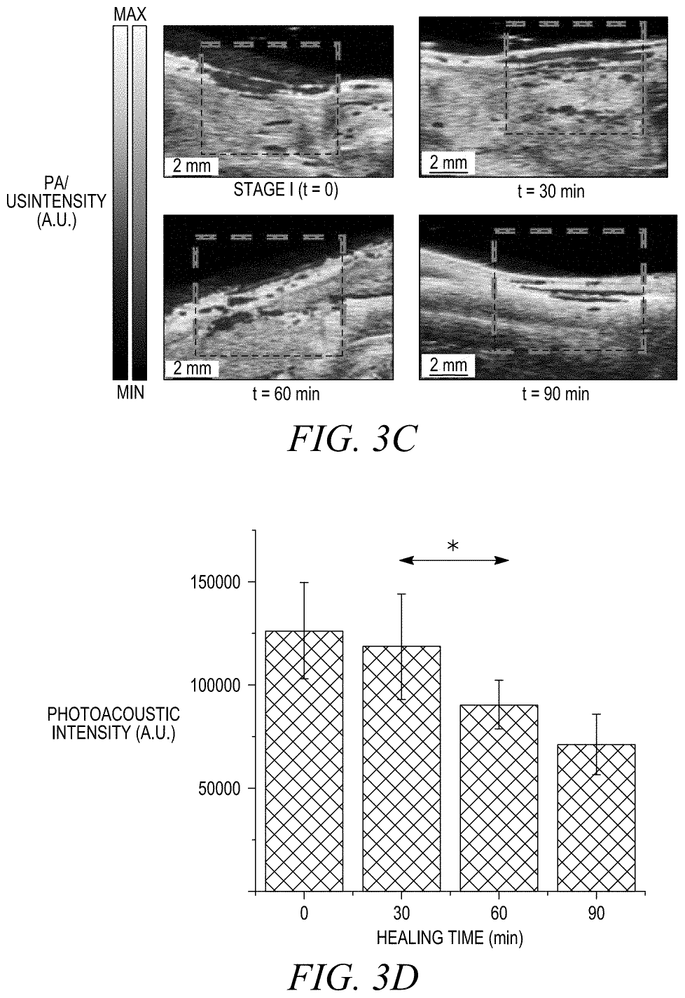

FIG. 3A, which illustrates the photoacoustic intensity as a function of ulcer stage, shows significant changes in photoacoustic intensity between baseline (no pressure ulcer) and all four stages (p<0.05). We found a 4.4-fold increase in photoacoustic intensity at stage I in comparison to the baseline. FIG. 3B quantifies the photoacoustic depth effect of the pressure ulcer on the skin and underlying tissues at different stages.

We measured photoacoustic ulcer depths of 0.38±0.09 mm, 0.74±0.11 mm, 1.63±0.4 mm, and 2.7±0.11 mm for stages I, II, III, and IV, respectively. There were significant differences between each stage (p<0.05). We also monitored the animal 30, 60, and 90 minutes after stage I to simulate healing/therapy.

FIG. 3C shows ultrasound/photoacoustic images at 0, 30, 60, and 90 minutes after stage I to simulate healing/therapy. The dotted rectangles show the regions of interest (ROIs). FIG. 3D shows the photoacoustic intensity as a function of healing time and thus quantifies the photoacoustic intensity effect of healing procedure. We observed significant decrease in photoacoustic intensity after 60 minutes (p<0.05).

These initial data were collected with a laser-based scanner; however, as noted above, these systems are bulky, delicate, and expensive. More recently, LED-based photoacoustic imaging system have offered important improvements in size, cost, and stability. Thus, we also evaluated these ulcers with LED-based photoacoustic systems.

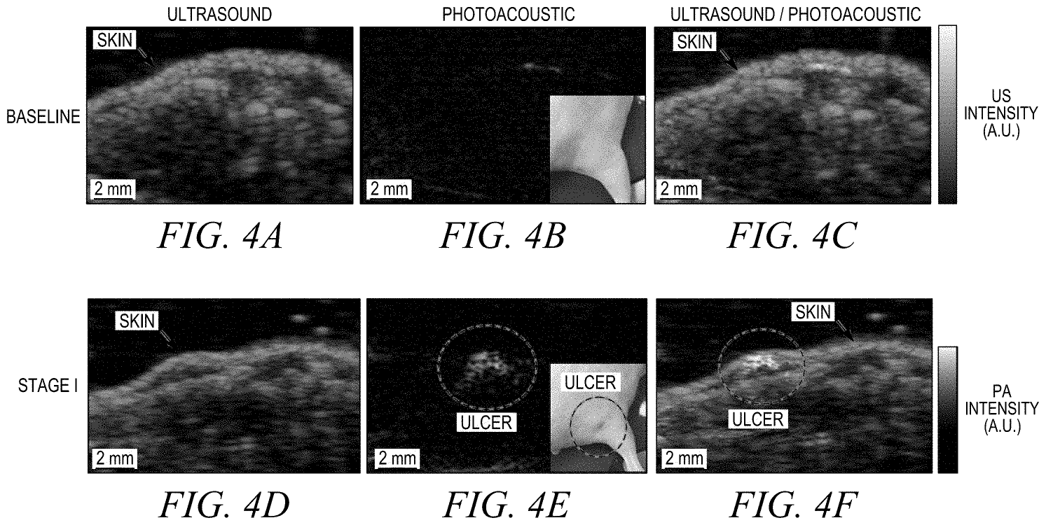

FIG. 4A shows a B-mode ultrasound image at baseline conditions when no pressure has been applied. FIG. 4B shows a B-mode photoacoustic image at baseline at the same position as in FIG. 4A. Minor photoacoustic signal is observed from the epidermis. The photographic inset in FIG. 4B shows the mouse in absence of ulcer. FIG. 4C shows a B-mode photoacoustic/ultrasound overlay at baseline conditions. FIG. 4D shows a B-mode ultrasound image at stage I and FIG. 4E shows a B-mode photoacoustic image at stage I at the same position as in FIG. 4D. We observed a 2.5-fold increase in photoacoustic intensity at stage 1 compared to baseline using the LED-based photoacoustic imaging system. The photographic inset in FIG. 4E shows the stage I ulcer. FIG. 4F shows a B-mode photoacoustic/ultrasound overlay at stage I. The image depth is 1 cm and the scale bars are 2 mm.

These preclinical results suggest that a LED-based photoacoustics imaging system has value for monitoring and staging these ulcers. Photoacoustic imaging is able to measure the status of underlying tissues without performing any invasive measurements such as histology.

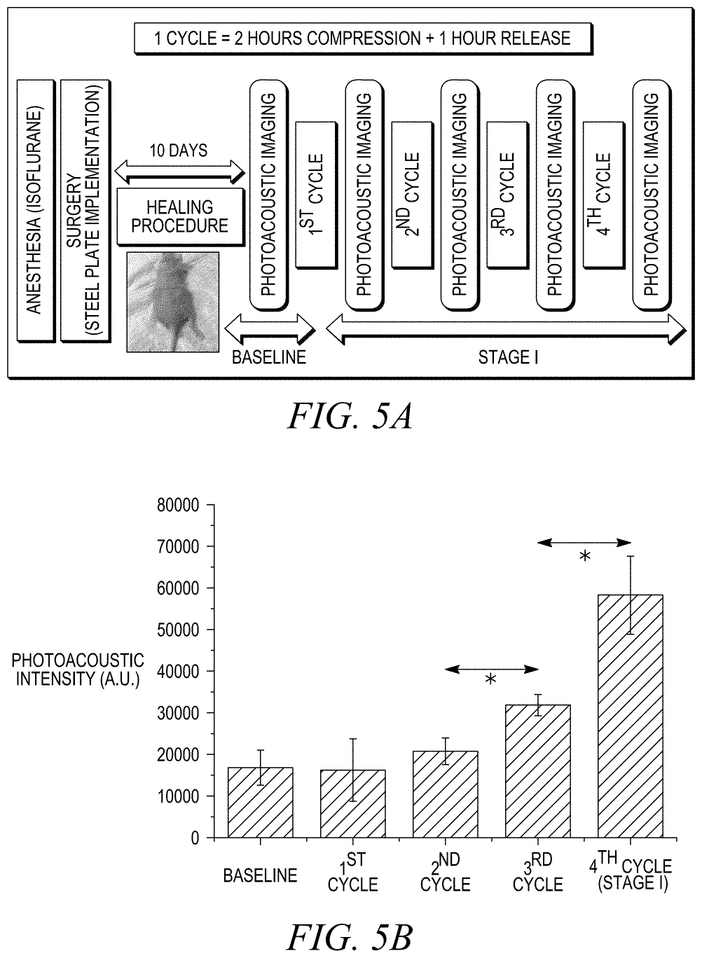

Our final goal was to monitor pre-stage I ulcers. Thus, we imaged after each of the cycles between baseline and stage I using the experimental procedure shown in FIG. 5A. There was no difference in photoacoustic intensity among the baseline, first, and second cycles. However, there was a significant increase in photoacoustic intensity between baseline/first cycle/second cycle and third/fourth cycle (p-value<0.05). FIG. 5B shows the photoacoustic intensity at each cycle. Therefore, photoacoustics can detect ulcers prior to the typical stage I classification. This imaging technique is sensitive enough to detect the mild physiological changes (Third and fourth cycles) that are not visible to the naked eye.

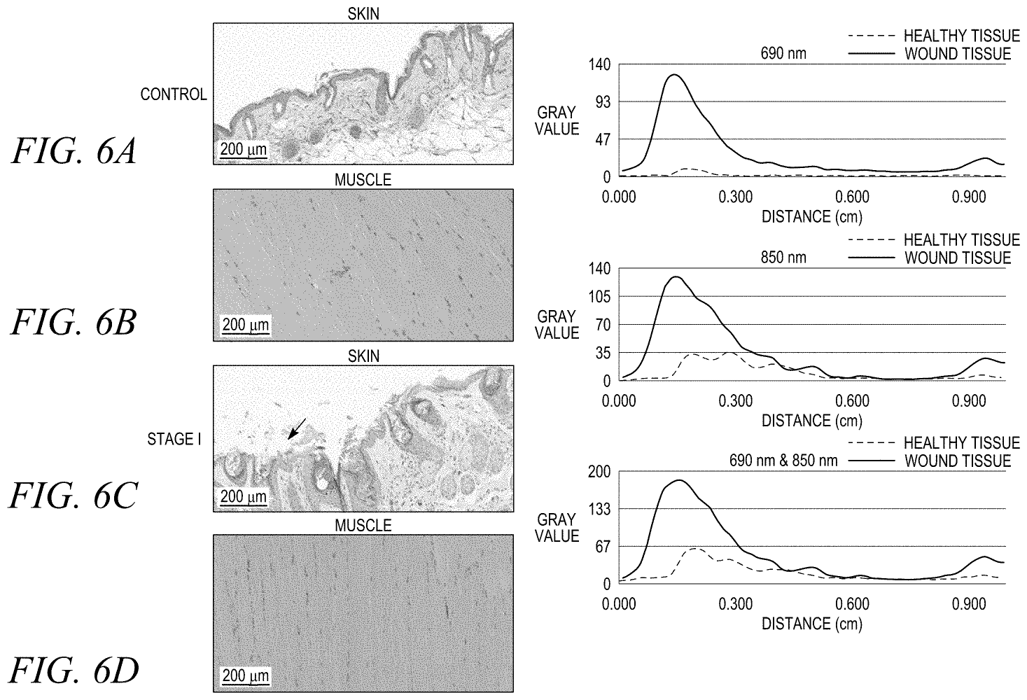

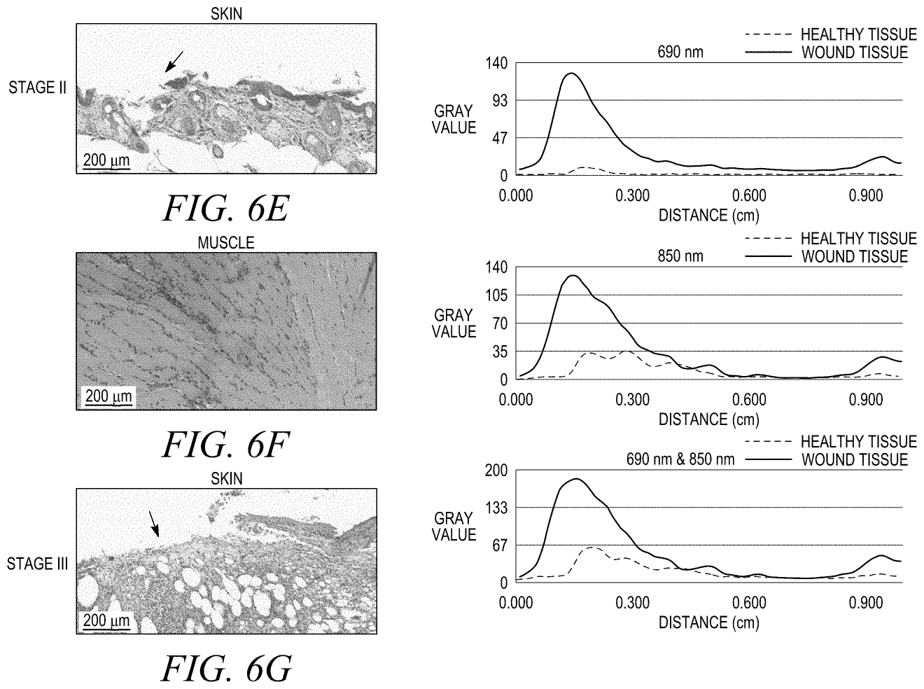

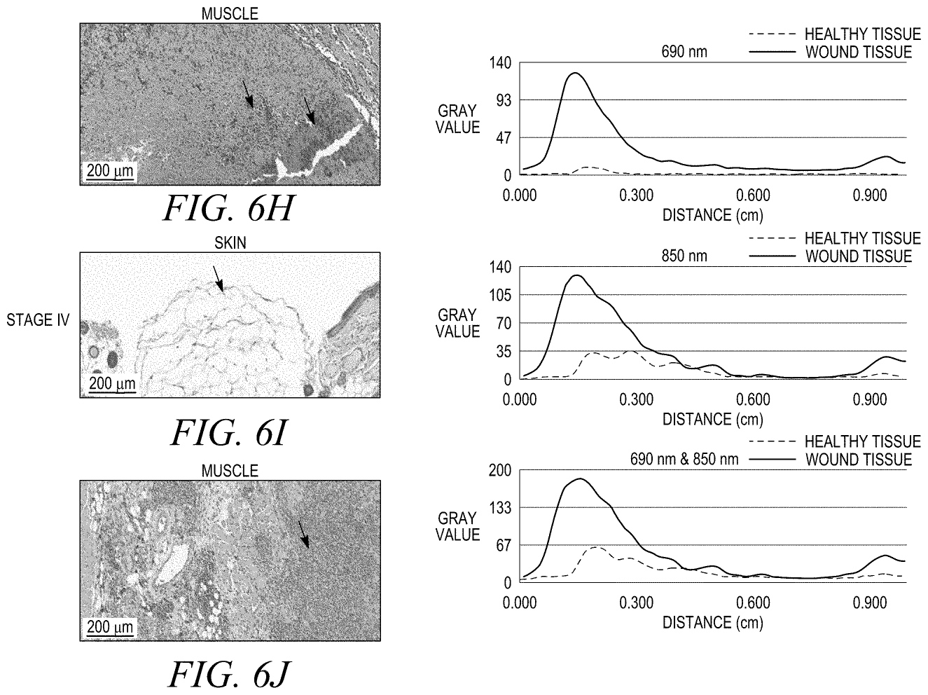

Finally, we validated the model with histology. FIGS. 6A and 6B are histological images of the skin and underlying tissue, respectively, for the baseline condition (no pressure ulcer). FIG. 6C shows a histology image of the skin at stage I. The arrow shows the superficial and epidermal skin loss in stage I. FIG. 6D shows the histology of muscle for the animals with stage 1—this is normal at stage I. FIGS. 6E and 6F show histological images of skin and muscle, respectively, at stage II. The structure of dermis and epidermis are now disrupted with mild necrosis on underlying tissues. FIG. 6G shows a histological image of skin in stage III of the pressure ulcer and FIG. 6H shows a histological image of muscle at stage III of the pressure ulcer. FIGS. 6G and 6 H demonstrate that stage III leads to full-thickness skin loss and necrotic areas in subcutaneous tissue layers. The histological image of skin in stage IV shown in FIG. 6I demonstrates that by stage IV all skin was removed. FIG. 6J shows a histological image of muscle at stage IV showing large necrotic regions of muscle. This confirmed the imaging data reflects dysregulated biology via the four-stage pressure ulcer from the Wassermann model.

Discussion

Pressure ulcers are debilitating and can significantly impair quality of life. They are associated with loss of pain sensation and disordered circulation. The gold standard to preventing pressure ulcers include regular patient turning/repositioning.

However, there are relatively few tools for molecular-level insight into when to reposition and who to reposition. This work describes a non-invasive, high resolution imaging technique that utilizes photoacoustic signal to detect pressure-induced tissue damage in a nude mouse model in vivo and may have utility in predicting the timing of repositioning.

We utilized both the intensity of photoacoustic signal as well as the depth of the photoacoustic signal to characterize ulceration. It has previously been shown that the photoacoustic signal changes can report the presence of inflammation and erythema. The photoacoustic depth penetration can distinguish the degree of injury analogous to conventional staging of pressure ulcers. Surprisingly, the signal intensity had less utility in differentiating ulcer stage; however, we can quantify the level of injury by calculating the position of high-intensity (bit depth >20 (8-bit images)). We used both laser and LED-based equipment. While the laser-based system has more power and higher resolution, LED-based equipment offers significant cost advantages that might aid in clinical translation. The results of LED-based photoacoustic imaging (shown in FIG. 4) could easily discriminate between different stages. More importantly, this equipment had sufficient sensitivity to detect the mild physiological changes associated with pre-Stage 1 lesions that are not visible to the naked eye (demonstrated in FIG. 5B). As demonstrated by FIGS. 1, 4 and 5, the value of imaging is that it offers a 3D map of the underlying tissue without invasive measurements such as histology.

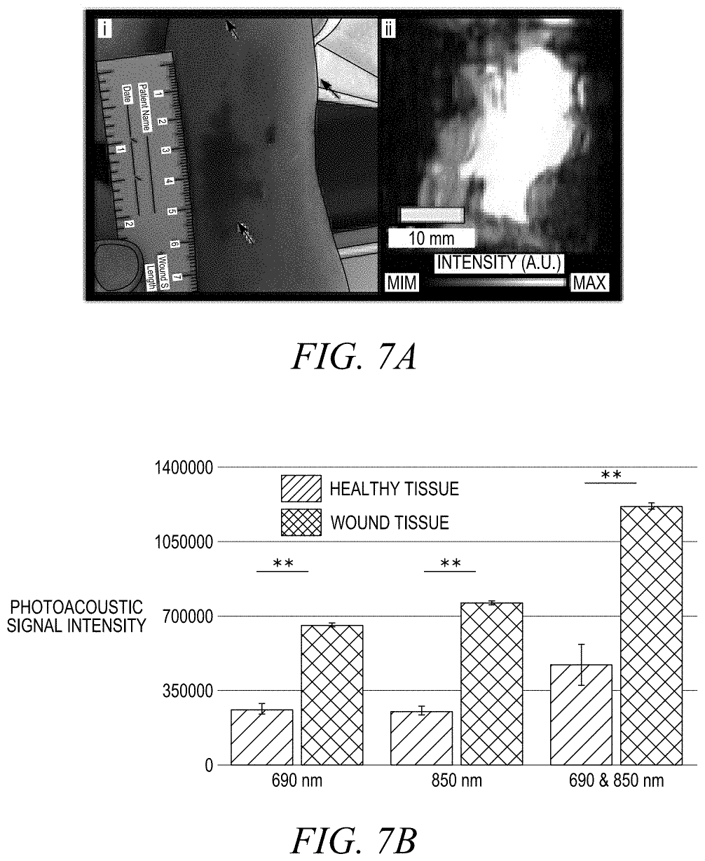

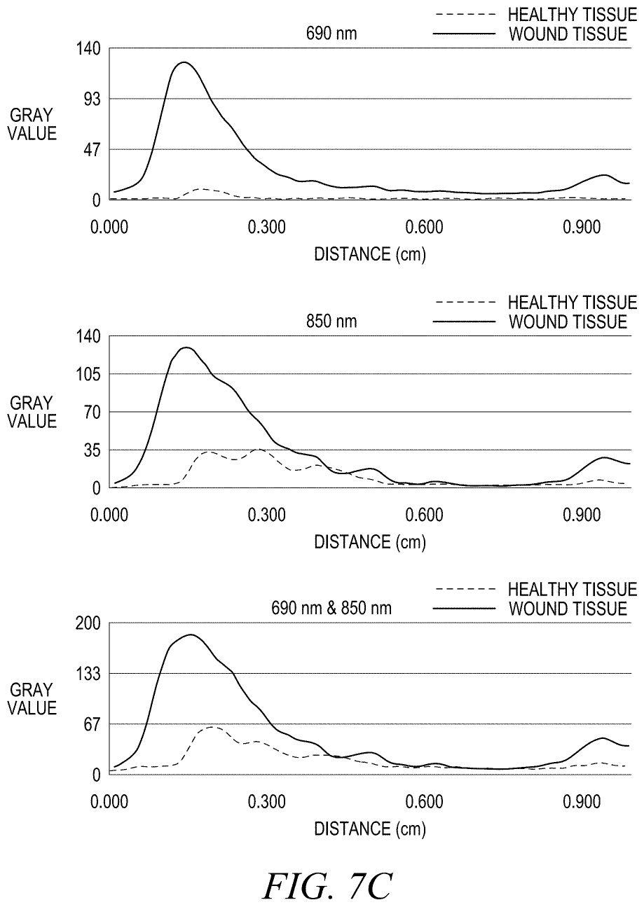

The murine model used here was confirmed histologically to present a phenotype consistent with human ulcers. To further demonstrate the applicability of this technique to skin diseases in humans, we imaged an approximately 2.5 cm×5 cm arterial insufficiency injury located on the left ankle (tibio-talar) joint in a 72-year-old male patient. Control tissue was determined to be the posterior tibia, healthy tissue located approximately 3 cm superior to the wound. FIG. 7A shows a photograph (i) and photoacoustic 3D mapping (ii) of the arterial insufficiency injury located at the left ankle (tibio-talar) joint in the patient. FIG. 7B presents a quantitative analysis of the arterial insufficiency injury and healthy adjacent tissue showing a statistically significant increase in photoacoustic signal intensity at 690 nm, 850 nm, 690 nm and 850 nm LED wavelengths. FIG. 7C shows a column average plot that was performed on all framed images and averaged to analyze mean photoacoustic signal intensity in the wound bed. The examination depth was limited to 9 mm (from the skin surface) to minimize the analysis of acoustic reflections. The column average plot shows a significant increase in mean photoacoustic signal and distinct photoacoustic signature in wound tissue compared to healthy tissue at 690 nm, 850 nm, 690 and 850 nm (combination) LED wavelengths (FIG. 7C).

The findings presented above in FIG. 7 collectively demonstrate that photoacoustic signal intensity and pattern can be used to assess the extent and development of a chronic wound in a human subject. Importantly, these findings illustrate the potential for monitoring real-time changes in a wound bed, particularly in response to chronic wound treatment, by evaluating the changes in photoacoustic single intensity and pattern. Additional findings have demonstrated that photoacoustic imaging can show laterally extending portions of a wound that are not visible to the eye on the skin surface.

### CONCLUSION

In summary, there is a clinical need for a noninvasive technique that can detect early tissue damage that would otherwise go unnoticed. We introduce photoacoustic imaging as a non-invasive tool that can be utilized to detect pressure ulcers before stage I. Here, we used a published protocol to produce pressure ulcers in nude mice. We observed significant changes in photoacoustic intensity even before stage I of pressure ulcer. We also showed that the stage of ulceration can be determined by quantifying the depth of photoacoustic signal from the injury. Importantly, the ability to detect and stage tissue damage can allow earlier and more effective treatment to be performed, including, by way of example, adjustment of a turning regimen, skin grafts and hyperbaric treatment based on the staging. We also observed that photoacoustic imaging can monitor ulcer healing, suggesting potential clinical value in monitoring therapeutic response.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. For example, while photoacoustic imaging has been demonstrated as being able to detect and stage pressure ulcers, more generally these same techniques may be used to detect a variety of different dermal or subdermal features such as pressure ulcers, diabetic foot ulcers, arterial insufficiency injuries, decubitus ulcers, diabetic ulcers, and insufficiency injuries. Furthermore, it is to be understood that the invention is solely defined by the appended claims.

1. A method of identifying a subdermal feature in a subject, comprising:
directing optical energy into a subject to photoacoustically generate ultrasonic waves from a dermal or subdermal feature in tissue;
receiving signals representing ultrasonic waves that are generated from the dermal or subdermal feature;
processing the signals representing the ultrasonic waves to generate image data of the dermal or subdermal feature; and
determining from the image data that the dermal or subdermal feature is a non-healing skin lesion selected from the group including a pressure ulcer, a diabetic foot ulcer, an arterial insufficiency injury, a decubitus ulcer, a diabetic ulcer, and an insufficiency injury.

2. The method of claim 1, wherein the determining includes determining from the image data that the dermal or subdermal feature is a non-healing skin lesion before the non-healing skin lesion is visible by eye.

3. The method of claim 1, wherein the determining includes determining that the non-healing skin lesion selected from the group including a pressure ulcer, a diabetic foot ulcer, an arterial insufficiency injury, a decubitus ulcer, a diabetic ulcer, and an insufficiency injury extends beyond a region that is visible by eye.

4. The method of claim 1, further comprising staging the non-healing skin lesion based at least in part on an effective depth of the subdermal feature.

5. The method of claim 1, wherein, when determining that the dermal or subdermal feature is a pressure ulcer, staging the pressure ulcer based at least in part on an effective depth of the subdermal feature.

6. The method of claim 5, further comprising also staging the pressure ulcer based at least in part on a photoacoustic intensity of the image data.

7. The method of claim 6, further comprising assigning a later stage to the pressure ulcer above a baseline as the effective depth of the subdermal feature increases.

8. The method of claim 4, wherein the non-healing skin lesion is a pressure ulcer.

9. The method of claim 4, wherein the non-healing skin lesion is a pressure ulcer and further comprising assigning a later stage to the pressure ulcer above a baseline as the effective depth of the subdermal feature increases and the photoacoustic intensity of the image data increases.

10. The method of claim 1, wherein the image data are B-mode photoacoustic images.

11. The method of claim 1, further comprising directing treatment of the dermal or subdermal feature based at least in part on the staging.

12. The method of claim 11, wherein the directing treatment includes adjusting a turning regimen of the subject based on the staging.

13. The method of claim 11, wherein the directing treatment includes skin grafts or hyperbaric treatment based on the staging.

14. The method of claim 1, wherein the determining includes determining that the dermal or subdermal feature is a pre-stage 1 pressure ulcer.

15. The method of claim 1, further comprising directing the optical energy and receiving the signal using an LED-based photoacoustic imaging system.

16. A method of monitoring ulcer healing in a subject, comprising:
directing optical energy into a subject to photoacoustically generate ultrasonic waves from an ulcer;
receiving signals representing ultrasonic waves that are generated from the ulcer;
processing the signals representing the first ultrasonic waves to generate image data of the subdermal feature; and
quantifying changes in photoacoustic intensity of the image data over time to confirm healing of the ulcer.

17. The method of claim 16, further comprising confirming that the ulcer is healing when a photoacoustic intensity of the image data increases over time.
